(19) United States
(12) Patent Application Publication
(10) Pub. No.: US 2018/0314797 A1
(43) Pub. Date: Nov. 1, 2018

(54) Cephalometric analysis and patient management software application for mobile devices

(71) Applicant: Rohit Madan, Al Juffair (BH)

(72) Inventor: Rohit Madan, Al Juffair (BH)

(21) Appl. No.: 15/582,820

(22) Filed: May 1, 2017

(51) Int. Cl. G06F 19/00; G06T 7/00; G06N 99/00; A61B 6/14; A61B 6/00; A61B 5/00

(52) U.S. Cl. CPC G06F 19/321; G06T 7/0016; G06F 19/325; G06N 99/005; G06T 2207/10116; A61B 6/501; A61B 5/0077; G06T 2207/30036; A61B 6/14

(57) ABSTRACT

A method and application executed through smartphone, tablet or similar devices using compatible operating systems for the cephalometric analysis of cephalograms and photographs of a patient to aid to orthodontic and cephalometric diagnosis, analysis and practice management tool. It helps its user to customize cephalometric analyses based on the values selected from different analysis. It also allows the users to receive, store, edit and access patient data on their devices.

### FIELD OF INVENTION

The invention generally relates to the field of cephalometric analysis, and particularly to a cephalometric analysis and practice management application to be executed through the smartphone.

### STATEMENT REGARDING FEDERALLY SPONSORED RESEARCH OR DEVELOPMENT

(Not Applicable)

### BACKGROUND OF INVENTION:

Cephalometric analysis refers to the study of the quantifiable data related to the dental and skeletal structures of the head. It is conducted by orthodontists and oral and maxillofacial surgeons for diagnostic, assessment and treatment planning purposes. It can help the clinician to accurately identify skeletal disproportions and dental malocclusions and also help in formulating an appropriate treatment plan.

In addition, cephalometric analysis can also assist in comparing before and after dental/ skeletal changes which may have occurred in response to orthodontic/surgical treatment and to also help predict future growth and development changes that may take place in the craniofacial complex.

A cephalogram is a radiographic projection of the skull taken by a cephalostat, an x-ray apparatus. Commonly the lateral cephalogram projection is used for the cephalometric analysis. The standardized nature of these projections has made it possible to establish the population norms. Cephalograms can be used to be measured and compared with previous projections of the patient or norms or observe the treatment progress.

There are various types of cephalometric analyses that describe the craniofacial complex. The orthodontists may use any cephalometric analysis of their choice and convenience. In traditional methods of analyzing a cephalogram, the radiograph is manually traced onto a frosted acetate sheet with the help of a lead pencil. As part of this tracing, the outlines of the skeletal and dental structures are drawn and the relevant cephalometric landmarks, lines and planes are identified. The tracing is then be used to record geometric measurements such as angles and linear distances.

The information about the cranial base, mandible, maxilla, dentition, and soft tissue helps the orthodontists to measure the various elements such as maxillary position, facial proportions, mandibular position, incisor position etc. Once the cephalometric data is collected, the orthodontists conduct the cephalometric analysis by comparing the cephalometric data with the standard cephalometric values as per the patient's race and ethnicity.

It consumes more time to use the traditional method to identify the cephalometric landmarks and conduct cephalometric analysis. Given the fast growth and advancement in the computer information technology, the cephalometry, similar to other fields, extensively uses the advanced computing devices and programs to simplify the methods and obtain more accurate results. The computer executed programs aid the orthodontists and experts in plotting the cephalometric landmarks on the cephalogram using an inputting device of the computer such as mouse and touchpad.

For an example, the prior art includes software programs marketed under the brand name Facad, OrthoGo, Dr. Ceph and others. Facad program allows orthodontists to perform cephalometric analysis of a patient and works on computers using the operating system Windows 7, 8, 8.1, Window Vista, Windows XP or MAC computer. It imports digital images such as JPEG, BMP, TIFF, DICOM, scanned x-ray films, cephalogram, frontal x-rays, panoramic x-rays, facial photos, cast models (2-D) and other images. It may be connected, and is adapted, to software programs for digital x-ray imaging, patient management systems, or PACS. Facad program also offers image calibration tools, image zooming, brightness, contrast and rotating tools.

The program traces the cephalometric landmarks and conducts the cephalometric analysis in the light of standardized analyses using the digital images. Facad program also offers treatment planning and superimposition tools. Facad program also allows the user to print the image, landmark tracing, cephalometric results, reports, copy, save or export the files.

Similarly OrthoGo offers tools for image viewing such as zoom, brightness and contrast. The software does also help to trace cephalometric landmarks including teeth, hard tissue and profile line. It also does cephalometric analysis and superimposing x-ray and profile photo, tracing and x-rays. In addition to the foregoing, the software is used for treatment planning, soft tissue prediction, cast model analysis and treatment objectives.

The software has certain standard analyses to be used in the cephalometric analysis of a particular patient. It allows creating the user's own analyses or customizing such standard analyses. The software is operated through the computer and is Windows 8/7/Vista, 32-bit and 64-bit supported.

Dolphin Imaging System produces an integrated product of three different programs. First takes image; second traces the cephalometric landmarks and offers superimposition tools; and third deals in the treatment simulation including diagnosing and planning.

Dr. Ceph, a cephalometric analysis software, is another example of the computer executed programs that offers the tools by which the user may morph an image, superimpose the x-ray onto the image of the patient, select cephalometric analysis and customize the user's own analysis or modify any analysis from the built-in analysis collection.

Further development taken place in the cephalometry has been witnessed upon the invention of the mobile tablet device applications for the cephalometric analysis. One Ceph and Cephalometric Analysis are some of the existing examples of the applications in the market. One Ceph offers some simple features including selecting cephalometric analysis, identifying cephalometric landmarks and deriving results. Cephalometric Analysis performs only landmark identification and cephalometric analysis.

The trend in the development of the cephalometric analysis methods and tools aims to provide the professionals with such technology that may save their time and effort, optimize the results and aid them in the treatment planning. To meet this end, the prior art fails to provide the professionals any handy and one-in-all means that may be used to perform cephalometric analysis, devise treatment planning and keep the data and files of the patient available all the time.

All prior art and technologies used in the cephalometric analysis, treatment planning and patient data management system require the professionals to use a specialized environment such as systems, computers and technological infrastructure installed or available in the office or laboratory premises. Since the prior art offers the programs operated through the desktop computers, it makes the user fail to utilize the opportunity to use their out of premises hours which might be used thanks to the advanced communication technology. The prior art does not address the needs of the user to access the patient files and data at all times. Though the cephalometric landmark tracing and analysis have become easy and simplified through some computer softwares and applications on tablet devices, there is much room for improvement that may make the operation of the cephalometric tracing and analysis programs further convenient by providing the necessary analysis tools in mobile smartphone devices.

### SUMMARY OF INVENTION:

The present invention aims to address the limitations and flaws of the prior art and to help fill the room for improvement. For the convenience of the reader, the present invention is referred to as “Application”. In the following space, the distinguishing features of the Application are described in detail. It is a smartphone application which supports the IOS and Android mobile operating systems. It also works well on tablet devices which utilize these operating systems. It is designed to allow the users to quickly, simply and accurately analyze their patients' cephalograms, image and photographs (hereinafter collectively referred to “image”) on their mobile devices by utilizing a friendly, intuitive and innovative user interface. The application is also designed to allow users to create custom cephalometric analysis, which can be shared with and used by other users. It also acts as a patient management tool and allows the users to receive, store, edit, access and share patient data conveniently at any time through their mobile devices.

After logging in, the Application displays the main menu listing leading, upon tapping, the user to any section they want to use. The image captured through the device camera or imported from the device or cloud storage is cropped to fit the further processes.

Next step following the cropping the image is the selection of an analysis from the collection of the popular analyses ordinarily used in the orthodontic industry. The user may use any analyses on the basis of which the cephalometric analysis for the patient may be conducted. Thus, once the user has selected an analysis, the next screen displays the tools for the calibration of the image. The calibration is done by matching the linear markings on the application's virtual ruler with those on the cephalogram ruler. The user is able to use zoom tools and pinching gesture tool on both these rulers to help in accurately completing the calibration process.

The Application allows the user to use a double-tapping gesture on the Application screen to identify and mark the cephalometric landmarks on the image or cephalogram through the pinpoint tool and, when marked, it shows the name of each cephalometric landmark. The Application contains an extensive zoom, brightness, contrast and undo tools that help the user to mark the landmarks with more accuracy and precision. The Application draws lines and angles between specific points as per the requirements of the selected analysis. The required hard and soft tissue outlines are identified by the software and traced automatically with the help of computer vision and machine learning technologies. Each landmark is described with a text and with a diagrammatic illustration for the user to understand more about the landmark and its ideal position on the image.

By this time, the input data required by the Application to process the cephalometric analysis is ready and by entering the data, the user is shown the cephalometric analysis results with comparative values, such as the values analyzed with reference to the patient who is subject to the cephalometric analysis and the standard values. It also shows whether the values are high or low compared to the normal as well as a provisional diagnosis for each value.

The user is also able to select specific variables from various analyses available in the application to create a custom analysis which can then be named and shared by the user with other users of the Application for their use. Furthermore, the users can also alter the normal values for each analysis to create custom analyses that can relate to specific population norms.

The Application allows the user to save the cephalometric analysis results, cephalogram, images, treatment progress, notes and other patient data in a new folder on the device storage. Furthermore, the application allows the user to convert the cephalometric analysis results to a PDF file.

### BRIEF DESCRIPTION:

Brief Description of drawing:

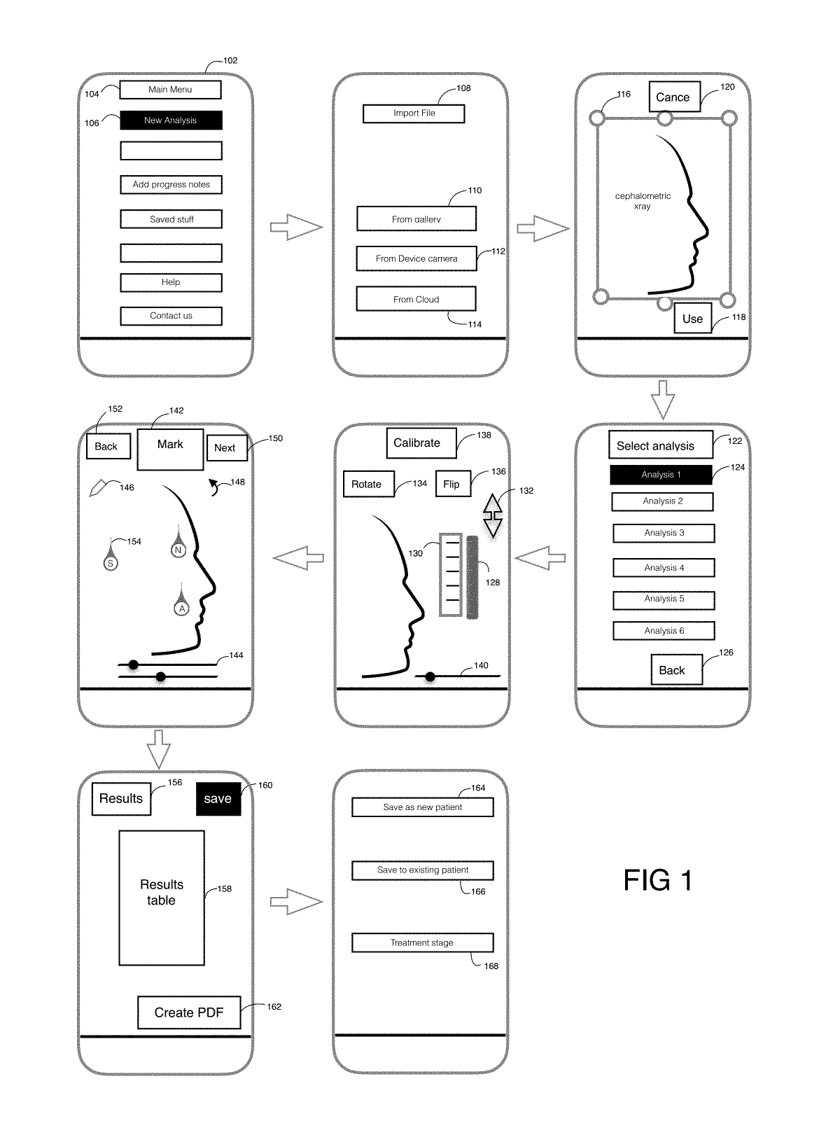

FIG. 1 shows how the application flows to conduct a cephalometric analysis.

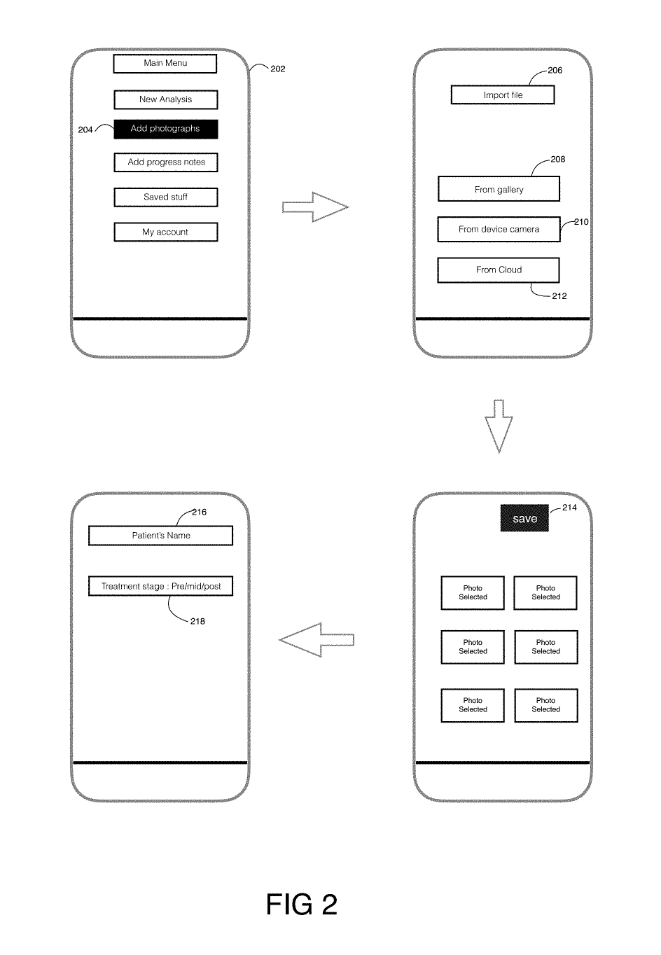

FIG. 2 describes the method to add and to save an image in the Application.

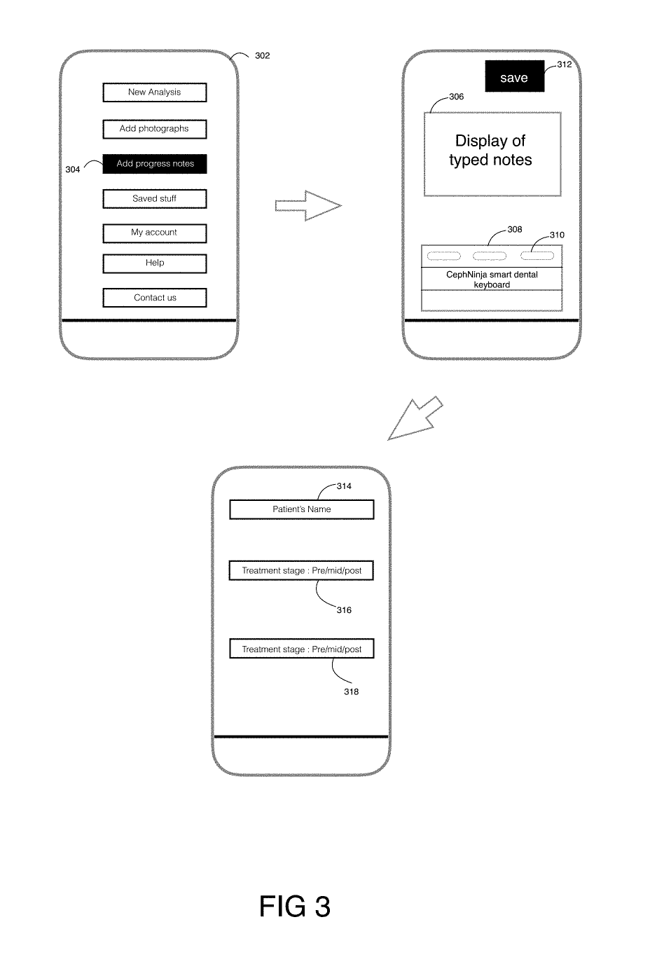

FIG. 3 describes how the application saves progress notes about the patient treatment.

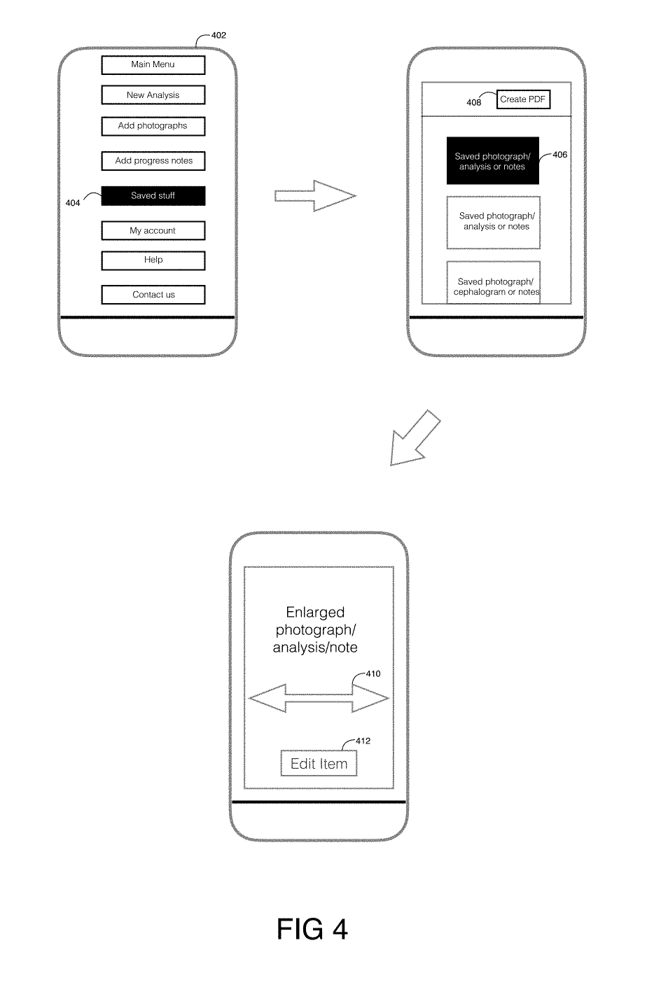

FIG. 4 explains the method of making use of the cephalogram or photo, progress notes or cephalometric analysis already saved in the Application.

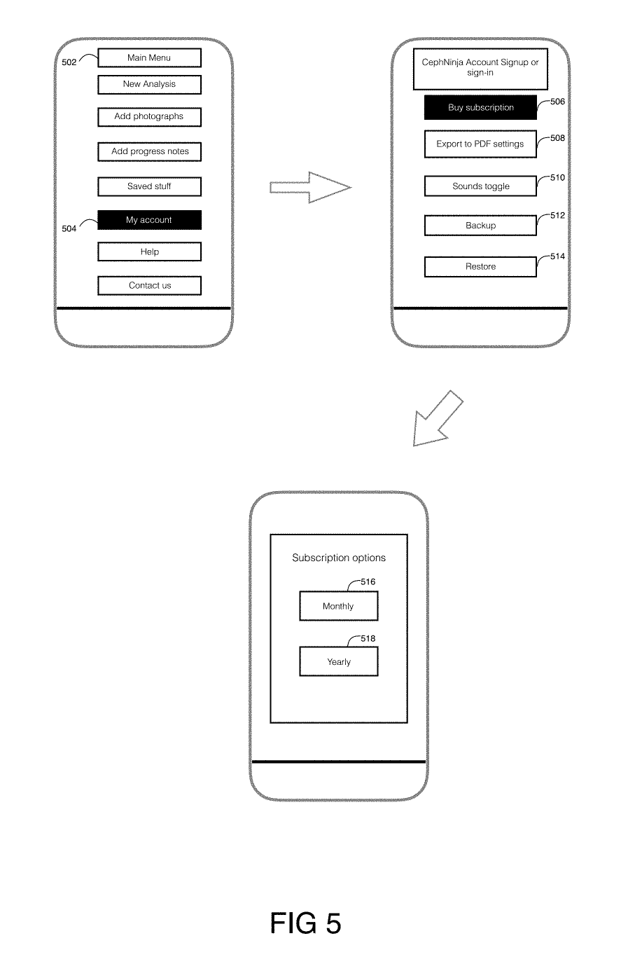

FIG. 5 explains the method of creating user account with the application and purchasing the subscription.

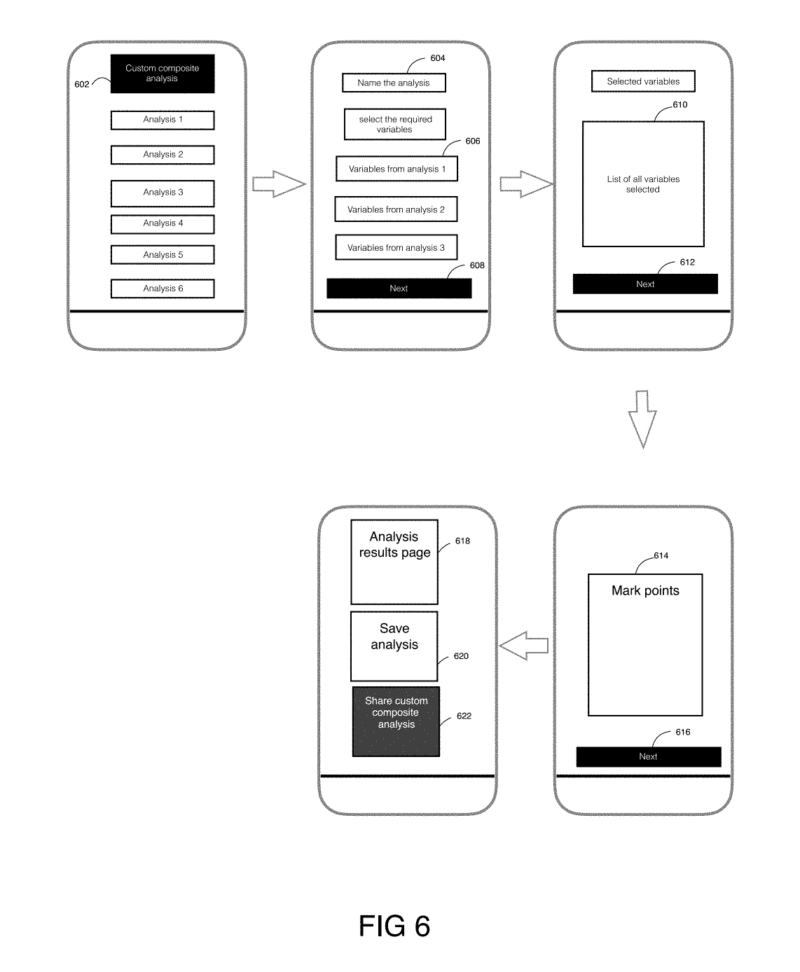

FIG. 6 explains how to create a custom analysis using variables from various the existing analyses.

### DETAILED DESCRIPTION OF DRAWING:

FIG. 1 shows how the application flows to conduct a cephalometric analysis. Firstly, a user interface screen of the smartphone 102 displays the main menu listing 104 which includes New Analysis 106, Add Photographs, Add Progress Notes, Saved Stuff, and My Account. The main menu listing 104 leads the user of the application to any section they want to use. Secondly, Tapping New Analysis 106 shows another section Import File 108 containing From Gallery 110, From Device Camera 112 and From Cloud 114. The user can import the image from Gallery of the device or by capturing a photo with the help of the camera of the device using From Device Camera or from the cloud storage using From Cloud button. Thirdly, once the photograph or cephalogram is imported from any of the available options i.e. Gallery 110, Device Camera 112 and Cloud 114, the cephalogram appears within a crop box 116. The crop box 116 allows the user to cut the image from all or any sides of the image to sever the irrelevant part of the cephalogram to improve its framing or the subject matter may be accentuated.

When the image has been cropped, the user needs to tap Use 118 situated at the right corner of the screen bottom that will bring the user to Select analysis section 122. If the user doesn't want to use the cropped image, they have an option to tap Cancel 120 which will get them back to the place where the image was originally imported from.

Next step following the cropping is the selection of an analysis page 122 that allows the user to select any analysis 124 from the collection of the popular analyses typically used in the orthodontic industry. The collection has two kinds of analyses such as cephalometric and photographic analyses. The user may use either of the analyses as per their suitability to a particular cephalogram or image. The analysis page also has a back option 126, which allows the user to go back to previous screen.

For the implementation of the principles of the selected analysis and for obtaining the outcomes of the cephalometric analysis accurately, the application must be provided with the exact dimensions and measurements of the cephalogram. Thus, when the user selects an analysis, the next display shows the tools for calibration i.e. a virtual ruler 128 and a cephalogram ruler 130. The virtual ruler 128 is a tool of this Application that appears on the screen in red color and the cephalogram ruler 130 is a ruler that is normally available as part conventional cephalograms. The virtual ruler 128 and cephalogram ruler 130 are aligned with each other matching their respective units on the touch screen of the Smartphone. For an example, when a cephalogram of a patient is imported, cropped and required to be calibrated, the user will need to drag the virtual ruler 128 to align it with the cephalogram ruler 130. If the user uses 10S, the virtual ruler 128 and the cephalogram ruler 130 may be zoomed in and out with the help of pinch or stretch motion 132 on either of the virtual ruler 128 or the cephalogram ruler 130, where applicable.

In addition to this, the Calibration section provides the option of Rotate Ruler 134 and Flip Cephalogram136. The user may rotate the rulers and flip the cephalogram by tapping Rotate ruler 134 or Flip 136 options available on the horizontal bar located at the top of the screen below the title Calibrate 138. Once both the rulers are brought parallel to each other, their dimensions and other measurements are matched by zooming either of them if required. In case of Android version, a sliding tab 140 is used to zoom in or out the virtual ruler 128 or the cephalogram ruler 130.

The next step after calibration is the identification of cephalometric landmarks. The Application displays the Mark Points page 142 with title, next 150 and back 152 option on the title bar. On the image, the Application allows the user to pinpoint as many points and landmarks as required for the analysis of the cephalogram or image. The user may double tap the screen to drop one point on the cephalogram or image. The points are shaped like a map pin and show the name of each cephalometric landmark 154 for identification. These points can be dragged to anywhere on the image. The Application contains an extensive zoom-in function that works with a pinch gesture and helps the user to mark the landmark with more accuracy. The Application draws lines between specific points as per the requirements of the selected analysis. Computer vision and machine learning technologies enable the app to automatically identify and draw hard and soft tissue outlines on the cephalogram. Each cephalometric landmark is described with a text and with a diagrammatic illustration for the user to understand more about the cephalometric landmark and its ideal position on the image.

Mark Points page contains a brightness/contrast 144 sliding tab and a CephNinja Edge feature button 146 both of which allow the user to change the contrast and look of the cephalogram or image to make the user able to better identify the cephalometric landmarks. With these features, essentially the outlines of the cephalogram internal structures become more prominent and easy to identify. The mark points 142 section also contains the Undo 148 option to reach to the last retrospective act with reference to the cephalometric landmarks. For example, the user makes an omission as to a point marked, the omission may be reversed by tapping the option Undo 148.

By the time the cephalometric landmarks are identified, the input data that the application requires to process is ready. A back button 152 allows user to go back to the previous screen. Tapping the next 150 option leads the user to the Results page 156 that shows the cephalometric analysis results. The cephalometric analysis results table 158 includes the normal values for a particular analysis as well as the measured values. It also shows whether the values are high or low compared to the normal as well as a provisional diagnosis for each value.

The Application allows the user to save the cephalometric analysis results in a new folder using the device storage by tapping Save 160. The user can create and save a PDF file by clicking on the create PDF button 162. Furthermore, clicking on save allows the user to save the analysis as a new patient name 164 or choose from an existing saved patient 166 and also enter the treatment stage 168 of the analysis.

FIG. 2 describes the method to add to and save a photograph in the application. A user interface screen of the smartphone 202 shows the main menu listing containing New Analysis, Add Photographs 204, Add Progress Notes, Saved Stuff, and My Account. On the main menu listing, the user may use any section of their choice. Tapping Add Photograph 204 shows another section Import File 206 containing From Gallery 208, From Device Camera 210 and From Cloud 212. The user can import the image from Gallery of the device 208 or by capturing a photo with the help of the camera of the device using From Device Camera 210 or from the cloud storage using From Cloud button 212.

Once the source of the image has been tapped, the screen shows the image files present in the folder of Gallery, Camera or Cloud Storage, as the case may be. The user may select any images of their choice and tap Save 214 to save the cephalogram or photo. Upon tapping the Save, the next screen appears that allows the user to save the images on a patient name 216 along with the treatment stage 218 as pre, mid or post treatment.

FIG. 3 describes how the application saves progress notes about the patient treatment. A user interface screen of the smartphone 302 displays the main menu listing from which Add Progress Notes, a link button, 304 is tapped to add the progress notes. The next screen allows the user to type, view or make any changes to the progress notes on the display of typed notes section 306 and touch screen dental smart keyboard 308 containing a regular keyboard along with pre-defined text tabs 310 that contain dentistry and orthodontic related terms and words. These tabs can be clicked which allow the user to type frequently used dental and orthodontic terminologies quickly and easily. Once the progress notes has been typed and made final, the user taps Save 312 to save the progress notes upon which the next display allows the user to save the notes with reference to a new patient name 314 or an existing patient along with the description as to the stage of the treatment 316.

FIG. 4 explains the method of accessing, cephalograms, progress notes or cephalometric analysis already saved in the application. Firstly, a user interface screen of the smartphone 402 displays the main menu listing which includes, among other link buttons, Saved Stuff 404 leading the user of the application to the following section where the user may open any of the saved files 406 including saved analysis, patient notes or images. On the top of the page, Create PDF 408 allows the user to export the saved file 406 to PDF file. The user may have an enlarged view of the saved files 406 using swipe arrow 410. Furthermore, the saved files 406 may be edited using the option Edit item 412.

FIG. 5 explains the method of creating user account with the Application and purchasing the subscription. The user needs to tap My Account 504 from the ‘main menu listing 502 to proceed to the next page where the user can login if the user has already created user account or sign-up to create new user account. The same page carries the link buttons Buy Subscription 506, export to PDF setting 508, sounds toggle button 510, Backup data 512 and Restore 514.

The export to PDF setting link 508 allows the user to adjust the settings related to the PDF files which are created by the application. The sound toggle button 510 allows the user to switch the application sounds to go on or off. The backup 512 allows for backing up the user created data and save it as a compressed file, and the restore 514 option allows the user to open a compressed file and import the data that was saved on it to the application. Upon tapping the Buy Subscription link button 506, the user is led to the next page showing Subscription Options—Monthly Subscription 516 and Yearly Subscription 518.

FIG. 6 deals with the method of creating the custom analysis based on the selection of the specific variables of the different analyses available in the application. Firstly, the user needs to tap Custom Composite Analysis 602 from the analysis listing page that will lead the user to following page showing the various options including Name the analysis 604 to name the custom analysis, Variable from Analysis 606 to select the variables to be used in the custom analysis, Next 608 to proceed to further next page to view the list of selected variables 610.

Tapping Next 612 shows the user the page where the cephalometric landmarks are marked 614 in the same manner as have been discussed in case of FIG. 1, and tapping Next 616 again gives the custom analysis results 618 which may be saved by tapping Save Analysis 620 for future use. The saved custom composite analysis may be shared with other users using Share Custom Composite Analysis 622.

1. A method and application for the cephalometric analysis of cephalograms and photographs of a patient to aid to orthodontic and cephalometric diagnosis, analysis and practice management tool executed through smartphone or similar technology device.

2. A method and application claim 1, wherein a photograph of the patient may be captured using the device camera.

3. A method and application claim 1, wherein the image or cephalogram of the patient may be imported from the device or cloud storage.

4. A method and application claim 1, wherein the photograph or cephalogram may be cropped.

5. A method and application claim 1, wherein an analysis from the database of the popular cephalometric analysis from the application may be selected to be applicable to the cephalometric analysis.

6. Method and application according to the claim 1, wherein the application offers a feature whereby the user can select specific variables from various analyses available in the application to create a custom analysis which can then be named and shared with other users for their use.

7. Method and application according to the claim 1, wherein the application offers a tool whereby the user can alter the normal values for each analysis to create custom analyses that can relate to specific population norms.

8. A method and application claim 1, wherein the photograph or cephalogram may be calibrated using virtual ruler and radiograph ruler.

9. A method and application claim 1, wherein double tap gesture is used to drop landmark pinpoints on the image.

10. A method and application claim 1, wherein the cephalometric analysis results are displayed on the screen.

11. A method and application claim 1, wherein the photograph may be captured through the device camera.

12. A method and application claim 1, wherein the photograph or cephalogram may be zoomed in or zoomed out while being calibrated or when the cephalometric points are being marked.

13. A method and application claim 1, wherein the application uses computer vision and/or machine learning technologies to automatically identify, mark and trace cephalometric landmarks and anatomic structures on the cephalogram.

14. A method and application claim 1, wherein the brightness of the photograph or cephalogram may be controlled during the cephalometric landmark points are being marked.

15. A method and application claim 1, wherein the result of cephalometric analysis, image or cephalogram or any progress note may be saved with reference to any patient.

16. A method and application claim 1, wherein the result of cephalometric analysis, treatment progress note or any saved files or photo may be converted to PDF files and may be printed.

17. A method and application claim 1, wherein the PDF files may be uploaded to any cloud storage to be accessible from anywhere.

18. The method and application claim 1, which allow the user to sign-up or sign-in and buy subscription to be able to use the same.

19. A method and application consisting of a feature to type notes relating to the cephalometric analysis, treatment progress notes and patient;

20. The method and application according to claim 19, wherein a smart orthodontic keyboard allows the users to quickly select the desired orthodontic and dental related terms for typing notes.
